(19) United States
(12) Patent Application Publication
(10) Pub. No.: US 2006/0105031 A1
(43) Pub. Date: May 18, 2006

(54) Accelerating recovery from trauma

(75) Inventors: Anthony E. Bolton, Dublin (IE); Arkady Mandel, North York (CA)

Correspondence Address: FOLEY & LARDNER LLP, 1530 PAGE MILL ROAD, PALO ALTO, CA, 94304, US

(73) Assignee: Vasogen Ireland Limited

(21) Appl. No.: 10/527,944

(22) PCT Filed: Sep. 15, 2003
(86) PCT No.: PCT/CA03/01407

§ 371 Date: Oct. 3, 2005

Related U.S. Application Data
(60) Provisional application No. 60/411,314, filed on Sep. 16, 2002.

(51) Int. Cl. A61K 9/127

(52) U.S. Cl. 424/450; 977/907

(57) ABSTRACT

Mammalian patients suffering from physical trauma, or about to likely suffer such trauma (by surgical treatment, or by suffering unanticipated accidental injuries, battle injuries or the like) are treated to lessen the severity of or accelerate the recovery from such consequently sustained trauma, by administering to the patient immune system-modifying entities, each comprising a body of a size similar to an apoptotic mammalian cell or apoptotic body, and having exposed on its surface phospho-glycerol groups, the entities being capable of being taken up by cells of the patient's immune system with accompanying beneficial effects including inhibition of pro-inflammatory cytokines and/or promotion of anti-inflammatory cytokines. The entities may be phosphatidylglycerol-presenting liposomes, generally of size 50 nanometers to 500 microns, and administered to contact a patient's immune system (e.g. intramuscularly) in amounts which affect but do not overwhelm the patient's immune system.

### FIELD OF THE INVENTION

This invention relates to therapeutic compositions and uses thereof in medical treatments and prophylaxis to lessen the effects of adverse medical conditions. More specifically, it relates to acceleration of recovery of a patient from the physical trauma of surgery and other wounds and injury conditions, and to methods of pre-conditioning the mammalian body so as better to withstand such physical trauma.

### BACKGROUND OF THE INVENTION

There is a continuing need to shorten the hospital stay of patients undergoing surgical procedures or treatment for physical trauma, which effectively means accelerating the rate of recovery of a patient from the trauma of surgery or other injuries. This applies both to patients undergoing pre-scheduled or elective surgery, to patients undergoing surgery as a result of accidental injury or treatment of an unforeseen medical emergency and to patients recovering from physical trauma having an inflammatory component. There is also a continuing need to better treat patients who suffer from myocardial infarction. Both for the comfort and rapid recovery of the patient, and for the benefit of health care economics, it is desirable to be able to accelerate the rate of recovery of a patient from such trauma.

It would also be desirable to be able to pre-condition a patient scheduled to undergo surgery, so that the patient would be better able to withstand the trauma associated with surgery, to lead to a more rapid recovery from trauma afterwards. It would also be advantageous to be able to precondition persons at risk of sustaining injury (battle troops, rescue personnel and the like) to enable them to recover more rapidly from such trauma.

### SUMMARY OF THE INVENTION

The present invention is based upon the novel appreciation of the role played by the up-regulaton of anti-inflammatory cytokines and/or the down regulation of inflammatory cytokines in a patient's body, and by improved endothelial function, on the mammalian body's wound process of recovery from physical trauma such as from surgery and other wounds. The natural process of apoptosis (programmed cell death) leads to the upregulation of anti-inflammatory cytokines and the down-regulation of inflammatory cytokines in the mammalian body, as well as improvements in endothelial function. The present invention provides a process of accelerating the recovery of a patent from physical trauma (surgical or accidental), and a process of pre-conditioning to accelerate the recovery from subsequently experienced such trauma, which mimics the apoptosis process of the mammalian body and takes advantage of the beneficial effects flowing from apoptosis in vivo, to effect such processes.

According to one aspect of the present invention, there is provided for use in the preparation of a medicament for treating a mammalian patient suffering from physical trauma, or treating a mammalian patient at risk of suffering such trauma (by surgical treatment, or by suffering unanticipated accidental injuries, battle injuries or the like) to lessen the severity of and/or accelerate the recovery from such trauma, of an effective immune system modifying amount of immune system-modifying entities, each comprising a body of a size similar to an apoptotic mammalian cell or apoptotic body, and having exposed on its surface phospho-glycerol groups, the entities being capable of modulating the patents immune system with accompanying beneficial effects including inhibition of pro-inflammatory cytokines and/or promotion of ant-inflammatory cytokines.

### THE PREFERRED EMBODIMENTS

One preferred category of such entities, the use of which in treatment of trauma and preconditioning against trauma constitutes a preferred embodiment of the present invention, is biocompatible synthetic entities such as biocompatible beads, comprising:

a three-dimensional head portion of size in its largest dimension of from 50 nanometers to 500 microns;

a plurality of tail portions bonded to each said head portion, the tail portions having:
phospho-glycerol end groups capable of modulating the appropriate receptors on antigen-presenting cells,
and chemical spacer groups of at least 3 linear carbon atoms, the spacer groups being bonded at their proximal ends to the respective head portion, and at their distal ends to the phosphate of the phospho-glycerol group.

The phospho-glycerol groups forming the end groups of the entities used in this embodiment of the invention have the general formula: 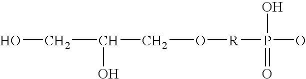
in which R represents C1-C4 straight chain or branched alkylene, alkylene-oxy, alkylene-thio, alkylene-amine, phenyl, iodo-substituted phenyl, and 5-membered N-heterocyclic groups, with the proviso that they interact with appropriate receptors on antigen-presenting cells.

In a particularly preferred embodiment, the tail portions of the entities such as derivatized beads have the chemical formula: 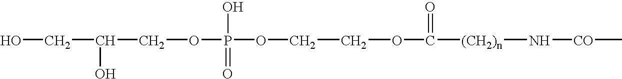
where n is an integer from 4-10, the amide end group being bonded to the head portion surface of the bead.

The term “beads” as used herein is intended to mean substantially any biocompatible body, solid, semisolid or hollow, shape-retaining and typically but not exclusively spheroidal, cylindrical, ellipsoidal including oblate and prolate spheroidal, serpentine, reniform, etc., and from about 50 nanometers to about 500 microns in diameter. They may be flexible or rigid. Preferred materials for their composition are polymethylmethacrylate, polyacrylate, polymethacrylate, glass, polystyrene, polyethylene, polypropylene and the like, of a grade approved for administration to mammalian patients.

The term “physical trauma” refers to trauma physically induced on a mammalian patient which in turn induces an inflammatory response. Such physical trauma includes, wounds, incisions, ischemia (whether induced by exogenous or endogenous factors), etc.

The phospho-glycerol end groups in entities used in this embodiment of the invention may be the distal end group of a phospholipid, namely phosphatidylglycerol, PG, the proximal end of which is attached to a body. These include particles, granules, microspheres or beads of biocompatible materials, natural or synthetic, such as polyethylene glycol, polyvinylpyrrolidone, polystyrene, etc., polysaccharides such as hydroxethyl starch, hydroxyethylcellulose, agarose and the like, as commonly used in the pharmaceutical industry. Some such suitable substances for derivatzation to attach the PG and, in the case of agarose, with PG attached, are commercially available, e.g. from Polysciences, Inc. 400 Valley Road, Warrington, Pa. 18976, or from Sigma Aldrich Fine Chemicals. The beads may be solid or hollow, or filled with biocompatible material. They are modified as required so that they carry PG molecules on their surfaces.

In a preferred embodiment, such phospho-glycerol carrying entities can be used for administration to patients about to suffer trauma involving wounds, e.g. patients about to undergo surgery or at high risk of suffering a wound as a result of imminent battle action, natural disaster etc., and will precondition the patient's body so as to accelerate the recovery from such subsequently encountered trauma. They will also have the effect of accelerating the recovery of a patient when administered to an already traumatized patient.

A further category of entities for use in another, particularly preferred embodiment of the invention is phosphatidylglycerol (PG) liposomes of the appropriate sizes referred to above, i.e., sizes resembling those of apoptotic mammalian cells or apoptotic bodies, and which have surface PG molecules. As a phospholipid, PG can form the membrane of a liposome, either as the sole constituent of the membrane or as a major or minor component thereof, with other phospholipids and/or membrane forming materials. Liposomes, or lipid vesicles, are sealed sacs, in the micron or sub-micron range, the walls of which consist of layers of suitable amphiphiles. They normally contain an aqueous medium.

The present invention contemplates the use, not only of those liposomes having PG as a membrane constituent, but also liposomes having non-PG membrane substituent but which carry on their external surface molecules of PG, e.g., chemically attached by chemical modification of the liposome surface, making the PG available for subsequent interaction with components of the patient recipient's immune system.

Preferred are liposomes constituted to the extent of 50% -100% by weight of phosphatidylglycerol (PG), the balance being phosphatidylcholine (PC) or other such biologically acceptable phospholipid(s). More preferred are liposomes constituted by PG to the extent of 65% -90% by weight. They are prepared from mixtures of the appropriate amounts of phospholipids as starting materials, by known methods.

Methods of preparing liposomes of the appropriate size are known in the art and do not form part of this invention. Reference may be made to various textbooks and literature articles on the subject, for example the review article “Liposomes as Pharmaceutical Dosage Forms” by Yechezkel Barenholz and Daan J. A. Chromeline, and literature cited therein, for example New, R. C., “Liposomes: A Practical Approach,” IRL Press at Oxford University Press, Oxford, England (1990), and Nassander, U. K., et al., In: “Biodegradable Polymers as Drug Delivery Systems” (M. Chasin and R. Langer, eds.) Marcel Dekker Inc., New York 1990, pages 261-338.

Such PG-carrying liposomes can be used for administration to patients about to suffer trauma involving wounds, e.g. patients about to undergo surgery or at high risk of suffering a wound as a result of imminent battle action, natural disaster etc., and will precondition the patient's body so as to accelerate the recovery of such subsequently encountered trauma. They will also have the effect of accelerating the recovery of a patient when administered to an already traumatized patient.

The successful application of the process of the present invention may be manifested in several ways, individually or collectively. The patient may manifest accelerated rate of wound healing, and/or more rapid decline of elevated body temperatures resulting from inflammatory cytokine action and fever as a result of wounding. In addition or in the alternative, the patient may evidence a more rapid recovery of joint mobility, e.g. following orthopedic surgery to replace or to repair a defective body joint (knee, hip, shoulder, etc). A greater survival rate of seriously injured patients is to be anticipated as a result of the use of the present invention. As a result, the duration of the hospital stay for the patient can be significantly reduced.

Another common manifestation of patients obliged to spend long periods in bed as a result of trauma from injury is the development of medical ulcers (decubitus or pressure ulcers). The processes of the present invention are indicated for acceleration of the healing of such ulcers, and indeed for treating and accelerating the healing of mammalian ulcers in general, and thereby further contributing to the shortening of the duration of a patient's hospital stay.

Without being limited by any theory, it is postulated that the sizes of the immune modifying entities used in the invention is such that they will be taken up by cells of the patient's immune-system in an apoptosis-mimicking fashion. In general, whatever type of entity is chosen, this means a size from about 50 nanometers to about 500 microns, more preferably from about 50 nanometers to about 500 nanometers.

The entities used in the process of the invention may be administered to the patient by any suitable means which brings them into operative contact with active ingredients of the patient's immune system. Preferably, the entities are constituted into a liquid suspension in a biocompatible liquid such as physiological saline and administered to the patient intra-arterially, intravenously, topically, transdermally (e.g. at a psoriatic site) or most preferably intramuscularly or subcutaneously.

A preferred manner of administering the entities to the patient is as a course of injections, administered daily, several times per week, weekly or monthly to the patient, over a period ranging from a week to several months. The frequency and duration of the course of the administration is likely to vary widely from patient to patient, and according to the severity of the trauma being treated or against which the patient is to be preconditioned. Its design and optimization is well within the skill of the attending physician. A schedule in which a patent receives daily injection on two consecutive days, 10-20 days prior to surgery, followed by a single, further injection 1-5 dyas prior to surgery, is especially recommended.

The quantities of entities to be administered will vary quite widely depending on the severity of the trauma it is intended to treat or against which is desired to precondition, and on the identity and characteristics of the patient. It is important that the effective amount of entities is non-toxic to the patient, and is not so large as to overwhelm the immune system.

When using intra-arterial, intravenous, subcutaneous or intramuscular administration of a liquid suspension of entities, it is preferred to administer, for each dose, from about 0.1-50 ml of liquid, containing an amount of entities generally equivalent to 1.0% -1000% of the number of cells normally found in an equivalent volume of whole blood or the number of apoptotic bodies that can be generated from them. Generally, the number of synthetic entities administered per delivery to a human patient is suitably in the range from about 500 to about 20×10⁹, preferably 10,000 to about 2×10⁹, as indicated by pre-clinical studies. Animal model results may not be truly representative of required numbers on a simple multiple of body weight, in an immune system modifying scenario.

Since the synthetic entities are acting, in the process of the invention, as immune system modifiers, in the nature of a vaccine, the number of such bodies administered to an injection site for each administration is a more meaningful quantitation than the number or weight of synthetic entities per unit of patient body weight. For the same reason, effective amounts or numbers of synthetic entities for small animal use may not directly translate into effective amounts for larger mammals on a weight ratio basis.

The invention is further described, for illustrative purposes, in the following specific examples.

### EXAMPLE 1

The invention can be demonstrated by experiments on laboratory rats, pretreating them with a course of injections of phosphatidylglycerol liposomes, surgically inserting temperature and heartbeat measuring probes into the pre-treated animals, and measuring their body temperature and other vital signs using the probes, as a measure of their recovery from the surgical major laparotomy reqired for insertion. The results are predictive of the effects on other mammals, including humans.

A total of 30 seven week old laboratory bred rats is separated into two groups of 15 animals each. Each animal of the test group A is administered, on day 1, day 2 and day 14 an intragluteal injection of 75% phosphatidylglycerol—25% phosphatidylcholine liposomes of size 100±20 nanometers, suspended in PBS, of volume 150 μL, each injection comprising 1,800,000 liposomes. Each animal of the control group B is similarly administered 150 μL of PBS containing no liposomes, on days 1, 2 and 14.

Four days after the completion of the injections, the animals are anaesthetized, and a telemetry probe is inserted surgically into the femoral artery of each animal. The telemetry probe (DATAQUEST LABPRO, from Data Sciences International) is a commercially available probe equipped with a radio transmitter, to permit heartbeat, systolic blood pressure, diastolic blood pressure and other signals to be received without further handling of the animals. An additional probe is surgically inserted into the peritoneal cavity of each animal, to measure body temperature.

Continuous daily recordings of body temperature, blood pressure and heart rate are made from each animal, for 10 days following the surgery. The group A test animals show a noticeably faster recovery of normal body temperature than the control group B, demonstrating a faster rate of wound healing and recovery from surgery in the test group.

### EXAMPLE 2

Surgery on mammalian patients commonly leads to secretion of large amounts of cytokines from the damaged tissue, with consequent weight loss in the patient. The speed with which the patient regains normal body weight is, accordingly, a measure of the rate of recovery from the trauma of surgery.

A group of 10 Balb C adult mice, of stable body weight (the “treatment group”) are given intramuscular Injections of 75% phosphatidylglycerol—25% phosphatidylcholine liposomes of size 100±20 nanometer, suspended in PBS. Each injection has a volume of 50 μL and contains approximately 600,000 liposomes. Injection takes place on days 1, 2 and 14. The mice are weighed on each day of injection.

On day 15, each mouse is subjected to laparotomy, and the wounds promptly stitched. The mice are weighed immediately 20 minutes after being stitched, and every 24 hours thereafter, for 7 days. Another group of 10 similar mice (the “control group”) are similarly weighed, subjected to laparotomy, wound stitching and weighing on the same schedule, but receive no injection of liposomes.

A noticeable and significant increase in the rate at which the treatment group of mice recover their preoperative body weight, as compared with the control group, is apparent.

All patents, patent applications, and publications previously cited above are herein incorporated by reference in their entirety.

1. Use in preparation of a medicament for treating a mammalian patient suffering from physical trauma, or treating a mammalian patient at risk of suffering such trauma to lessen the severity of and/or accelerate the recovery from such trauma, of immune system-modifying entities, each comprising a body of a size similar to an apoptotic mammalian cell or apoptotic body, and having exposed on its surface phospho-glycerol groups, the entities being capable of modulating the patient's immune system with accompanying beneficial effects including inhibition of pro-inflammatory cytokines and/or promotion of anti-inflammatory cytokines.

2. Use according to claim 1 wherein the entities are synthetic beads carrying phospho-glycerol groups.

3. Use according to claim 1 wherein said entities are PG liposomes.

4. Use according to claim 3 wherein the PG liposomes comprise from 50% to 100% PG by weight.

5. Use according to claim 1 wherein the bodies have a diameter of from 50 nanometers to 500 microns.

6. Use according to claim 1 wherein the unit dose is from about 500 to about 20×10⁹ entities.

7. A process of treating a mammalian patient suffering from physical trauma, or treating a mammalian patient at risk of suffering such trauma (by surgical treatment, or by suffering unanticipated accidental injuries, battle injuries or the like) to lessen the severity of and/or accelerate the recovery from such trauma, which comprises administering to the patient an effective immune system modifying amount of immune system-modifying entities, each comprising a body of a size similar to an apoptotic mammalian cell or apoptotic body, and having exposed on its surface phospho-glycerol groups, the entities being capable of being taken up by cells of the patient's immune system with accompanying beneficial effects including inhibition of pro-inflammatory cytokines and/or promotion of anti-inflammatory cytokines.

8. The process of claim 7 wherein said entities are phosphatidylglycerol liposomes.

9. The process of claim 7 wherein the phosphatidylglycerol liposomes have a size from 50 nanometers to 500 microns.

10. The process according to claim 7 wherein the entities are synthetic beads carrying phospho-glycerol groups.

11. The process according to claim 8 wherein the PG liposomes comprise from 50% to 100% PG by weight.

12. The process according to claim 7 wherein the bodies have a diameter of from 50 nanometers to 500 microns.
